(19) United States
(12) Patent Application Publication
(10) Pub. No.: US 2026/0348977 A1
(43) Pub. Date: Nov. 5, 2026

(54) Immunoassay for Detecting a N-Terminal Peptide of a Proteolytic Cleavage Product of Cartilage Intermediate Layer Protein-1

(71) Applicant: Nordic Bioscience A/S, Herlev (DK)

(72) Inventors: Signe Holm Nielsen, Frederiksberg (DK); Cecilie Hausgaard Møller, København SV (DK); Helena Port, Herlev (DK); Anne-Christine Bay-Jensen, København S (DK); Yi He, Virum (DK); Morten Karsdal, København Ø (DK)

(73) Assignee: Nordic Bioscience A/S, Herlev (DK)

(21) Appl. No.: 19/111,691

(22) PCT Filed: Sep. 14, 2023
(86) PCT No.: PCT/EP2023/075363

§ 371 Date: Mar. 13, 2025

(30) Foreign Application Priority Data
Sep. 14, 2022 (GB) 2213495.1

(51) Int. Cl. G01N 33/68; C07K 16/18

(52) U.S. Cl. CPC G01N 33/6887; C07K 16/18; C07K 2317/34; C07K 2317/92; G01N 2470/04; G01N 2470/10; G01N 2800/102; G01N 2800/105; G01N 2800/52

(57) ABSTRACT

Described herein are monoclonal antibodies that bind to an N-terminus sequence (SLNPDTGLWE (SEQ ID NO: 1), also referred to herein as CILP-MP) of cartilage intermediate layer protein-1 that is generated by cleavage by MMP-1, MMP-8, and MMP-12. Also disclosed are immunoassay methods and kits for detecting CILP-M in a patient sample, which methods and kits may be used for detecting and monitoring joint diseases and assessing the likelihood of patient response to drug treatment.

### FIELD OF INVENTION

The present invention relates to monoclonal antibodies that bind to an N-terminus sequence of cartilage intermediate layer protein-1 generated by cleavage by MMP-1, MMP-8, and MMP-12. The present invention also relates to methods of immunoassay for detecting said N-terminus sequence of cartilage intermediate layer protein-1 in a sample, such as whole blood, plasma or serum, from a patient and immunoassay kits suitable for carrying out said methods.

### BACKGROUND

Articular cartilage is a heterogenous tissue where cells are organized into a matrix. The matrix is formed by chondrocytes and its major constituents are fibril forming collagens, such as type I and III collagen and large aggregating proteoglycans (1). To form the cartilage matrix, other non-collagenous extracellular matrix proteins, such as the cartilage intermediate layer protein (CILP) are also present (2). CILP-1 is a glycoprotein, first identified in articular cartilage, and believed to have a role in cartilage scaffolding (2). It is secreted from articular chondrocytes (2), which are responsible for maintaining cartilage homeostasis by low-turnover remodelling of matrix proteins (3). A hallmark of rheumatic joint diseases is altered cartilage turnover, leading to dysregulated composition of the cartilage. During cartilage erosion, CILP-1 is secreted from articular chondrocytes and deposited into the cartilage extracellular matrix (2). CILP-1 has been shown to be cleaved in-vitro by various human proteases resulting in the generation of various fragments (4). It has been shown that synthesis of CILP was increased in early osteoarthritis (OA) cartilage (5) and associated with musculoskeletal disorders (6,7). In another study, CILP-1 was highly expressed in intervertebral discs and its expression increased in lumbar disc disease (8). An immune response to CILP has also been suggested to contribute to the pathogenesis of inflammatory joint destruction present in rheumatoid arthritis (RA) and OA (9). Moreover, CILP mRNA expression was observed in synovial tissue from patients with OA (10), indicating its expression is not restricted to articular cartilage. These findings suggest that CILP proteins may play a role in cartilage structure and rheumatic diseases.

### SUMMARY

The applicant has now discovered that CILP-1 is degraded by MMP-1, MMP-8, and MMP-12 to generate a neo-epitope having the N-terminus sequence SLNPDTGLWE (SEQ ID NO: 1) (referred to herein as “CILP-M” and/or “the target sequence”). The applicant has also discovered that CILP-M is generated and released into the circulation in-vivo, and has developed an immunoassay targeting CILP-M. Furthermore, the applicant has evaluated the biological value of CILP-M in joint-related diseases (RA, AS and OA) in two exploratory patient studies and has investigated the prognostic potential of CILP-M in anti-TNF-α treatment, and has shown that the measured levels of CILP-M in serum samples reflect disease activity.

Accordingly, in a first aspect the present invention provides a monoclonal antibody that specifically binds to the N-terminus amino acid sequence SLNPDTGLWE (SEQ ID NO: 1).

As used herein the term “N-terminus” refers to a N-terminal peptide sequence at the extremity of a polypeptide, i.e. at the N-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses (e.g. IgG1, IgG2, IgG3 or IgG4 in the case of human antibodies).

The CDR of an antibody can be determined using methods known in the art such as that described by Kabat et al. Antibodies can be generated from B cell clones as described in the examples. The isotype of the antibody can be determined by ELISA specific for IgM, IgG or IgA isotype (human or mouse), or subclass (human or mouse). The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In a preferred embodiment, the monoclonal antibody does not specifically bind to a peptide having the N-terminus amino acid sequence WSLNPDTGLWE (SEQ ID NO: 2).

In a preferred embodiment, the monoclonal antibody does not specifically bind to a peptide having the N-terminus amino acid sequence LNPDTGLWE (SEQ ID NO: 3).

In a preferred embodiment, the monoclonal antibody does not specifically bind to a peptide having the N-terminus amino acid sequence NPDTGLWE (SEQ ID NO: 4).

In a preferred embodiment, the monoclonal antibody is raised against a synthetic peptide having the N-terminus amino acid sequence SLNPDTGLWE (SEQ ID NO: 1). For example, the monoclonal antibody may be raised by: (a) immunizing a rodent (or other suitable mammal) with a synthetic peptide comprising the N-terminus sequence SLNPDTGLWE (SEQ ID NO: 1), which peptide may optionally be linked at its C-terminus to an immunogenic carrier protein (such as keyhole limpet hemocyanin); (b) isolating and cloning a single antibody producing cell; and (c) assaying the resulting monoclonal antibodies to ensure that they have the desired specificity.

In certain exemplary embodiments, the monoclonal antibody may preferably comprise one or more complementarity-determining regions (CDRs) selected from:

CDR-L1:
(SEQ ID NO: 5)
KASQNVGTDVV
CDR-L2:
(SEQ ID NO: 6)
SASYRYS
CDR-L3:
(SEQ ID NO: 7)
QHYDNYPLT
CDR-H1:
(SEQ ID NO: 8)
NYYIH
CDR-H2:
(SEQ ID NO: 9)
WISPGIVDTEYNEKFKN
CDR-H3:
(SEQ ID NO: 10)
RSAGSYGDFDY

Preferably the monoclonal antibody comprises at least 2, 3, 4, 5 or 6 of the above listed CDR sequences.

Preferably the monoclonal antibody has a light chain variable region comprising the CDR sequences:

CDR-L1:
(SEQ ID NO: 5)
KASQNVGTDVV
CDR-L2:
(SEQ ID NO: 6)
SASYRYS
CDR-L3:
(SEQ ID NO: 7)
QHYDNYPLT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 11)
KASQNVGTDVVWYQQRPGQSPKALMYSASYRYSGVPDRFTGSGSGTDFTL
TISNVQSEDLAEYECQHYDNYPLT

Preferably the monoclonal antibody has a heavy chain variable region comprising the CDR sequences:

CDR-H1:
(SEQ ID NO: 8)
NYYIH
CDR-H2:
(SEQ ID NO: 9)
WISPGIVDTEYNEKFKN
CDR-H3:
(SEQ ID NO: 10)
RSAGSYGDFDY .

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 12)
NYYIHWVKQRPGQGLEWIGWISPGIVDTEYNEKFKNKATLTADKSSRT
AYMQLSSLTSEDSAVYFCARRSAGSYGDFDY

Preferably, the monoclonal antibody comprises the light chain variable region sequence:

(SEQ ID NO: 13)
DIVMTQSQKFMSTSVGDRVSVTCKASQNVGTDVVWYQQRPGQSPKALM
YSASYRYSGVPDRFTGSGSGTDFTLTISNVQSEDLAEYFCQHYDNYPL
TFGAGTKLELK (CDRs bold and underlined;
Framework sequences in italics)
and/or
the heavy chain variable region sequence:
(SEQ ID NO: 14)
QVQLQQSGPELVKPGASVRISCKASGYTFTNYYIHWVKQRPGQGLEWI
GWISPGIVDTEYNEKFKNKATLTADKSSRTAYMQLSSLTSEDSAVYFC
ARRSAGSYGDFDYWGQGTTLTVSS

(CDRs bold and underlined; Framework sequences in italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In a second aspect, the present invention provides a method of immunoassay comprising:
i) contacting a patient sample with a monoclonal antibody in accordance with the first aspect of the present invention;
ii) detecting and determining the amount of binding between said monoclonal antibody and peptides in the sample.

In preferred embodiments, the method is a method for detecting and/or monitoring a disease in a patient and/or assessing the likelihood of the patient responding to a treatment with a drug to treat a disease, the method further comprising:
iii) correlating said amount of binding with values associated with normal healthy subjects and/or values associated with known disease severity and/or values obtained from said patient at a previous time point and/or with a predetermined cut-off value.

In preferred embodiments the disease is a joint disease. Preferably the joint disease is rheumatoid arthritis, ankylosing spondylitis or osteoarthritis.

In preferred embodiments the treatment with a drug to treat a disease is an anti-TNF-α treatment.

In preferred embodiments the patient sample is selected from blood, serum or plasma. Preferably the sample is serum or plasma.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to thereby determine the quantity of peptide to which the antibody specifically binds in the patient sample. In the Examples set out below, an ELISA method is used in which spectrophotometric analysis is used to measure the amount of binding both in the patient samples and when producing the calibration curve. However, any suitable analytical method can be used.

As used herein the term “predetermined cut-off value” means an amount of binding that is determined statistically to be indicative of a high likelihood of a disease or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or said particular severity thereof.

As used herein, the term “values associated with normal healthy subjects” means standardised quantities of binding determined by the method described supra for samples from subjects considered to be healthy, i.e. without disease; and the term “values associated with known disease severity” means standardised quantities of binding determined by the method described supra for samples from patients known to have disease of a known severity, for example a known likelihood of responding to a treatment.

In a third aspect, the present invention provides a method of treating a joint disease in a patient in need thereof, the method comprising:
(a) carrying out a method of immunoassay in accordance with the second aspect of the present invention on a sample from a patient in order to detect whether the patient has a joint disease and/or predict if the patient will respond to a therapy for a joint disease; and
(b) administering to the patient a medicament for the treatment of said joint disease if it is determined in step (a) that the patient has said joint disease or if it is determined that a patient is likely to respond to a treatment for the joint disease.

The medicament may be any medicament suitable for treating the joint disease in question. The medicament may for example comprise or consist of one or more topical medicaments, one or more systemic medicaments, or combinations thereof. Topical medicaments may for example be formulated as creams, foams, gels, lotions, or ointments for administration to the area or areas of joint requiring treatment. Systemic medicaments may for example be formulated for enteral or parenteral administration.

For example, where the joint disease is osteoarthritis, suitable topical medicaments may be selected from non-steroidal anti-inflammatory drugs (e.g. diclofenac or ketoprofen) or topical capsaicin; suitable systemic medicaments may be selected from pain killers (e.g. paracetamol or opioids); anti-TNF-α therapies (e.g. etanercept, infliximab, golimumab, adalimumab or apremilast); glucocorticoids (e.g. hydrocortisone or triamcinolone) or platelet-rich plasma.

Where the joint disease is rheumatoid arthritis, suitable medicaments may for example be topical medicaments which may be selected from non-steroidal anti-inflammatory drugs (e.g. diclofenac or ketoprofen) or topical capsaicin; suitable systemic medicaments may be selected from disease-modifying antirheumatic drugs (e.g. methotrexate, sulfasalazine, leflunomide, hydroxychloroquine); anti-TNF-α therapies (e.g. etanercept, infliximab, golimumab, adalimumab or apremilast); antibody therapies (e.g. rituximab or tocilizumab); pain killers (e.g. paracetamol or opioids); and glucocorticoids (e.g. hydrocortisone or triamcinolone).

Where the joint disease is ankylosing spondylitis, suitable medicaments may for example be selected from anti-inflammatory drugs (e.g. ibuprofen, phenylbutazone, diclofenac, indomethacin, naproxen or COX-2 inhibitors), disease-modifying antirheumatic drugs (e.g. methotrexate, sulfasalazine, leflunomide, hydroxychloroquine), anti-interleukin-6 inhibitors (e.g. tocilizumab or rituximab); interleukin-17A inhibitors (e.g. secukinumab and ixekizumab); janus kinase inhibitors (e.g. tofacitinib); and anti-TNF-α therapies (e.g. etanercept, infliximab, golimumab, adalimumab or apremilast).

In certain embodiments, step (a) of the method may comprise carrying out the method of immunoassay in accordance with the first aspect of the present invention on the sample from the patient to determine if the patient is likely to respond to treatment for a joint disease, and step (b) of the method may comprise administering to the patient a medicament for the treatment of said joint disease only if it is determined in step (a) that the patient is likely to respond to treatment.

For example, the method may involve administering a medicament if the likelihood of responding to a treatment is at or above a particular level. In one particular embodiment, the joint disease is ankylosing spondylitis, and step (b) comprises administering an anti-TNF-α therapy to the patient if it is determined in step (a) that the patient has a level of CILP-M in the sample that is associated with a likelihood of responding to the anti-TNF-α therapy, and administering an anti-TNF-α therapy if it is determined the patient is likely to respond to treatment.

In a fourth aspect, the present invention provides an immunoassay kit comprising a monoclonal antibody in accordance with the first aspect of the present invention, and at least one of;
a streptavidin coated well plate;
a biotinylated peptide SLNPDTGLWE-L-Biotin (SEQ ID NO: 15), wherein L is an optional linker;
a secondary antibody for use in a sandwich immunoassay;
a calibrator protein comprising the N-terminus amino acid sequence SLNPDTGLWE (SEQ ID NO: 1);
an antibody biotinylation kit;
an antibody HRP labelling kit;
an antibody radiolabelling kit; and
an assay visualisation kit.

The immunoassay kit according to the fourth aspect of the invention is, in particular, suitable for use in carrying out the method according to second aspect of the invention. Further preferred embodiments and features of the immunoassay kit according to the fourth aspect will therefore be apparent from the above discussion of the preferred embodiments of the method according to the second aspect.

### FIGURES

FIG. 1: Alignment and specificity of the CILP-M assay. A) A sequence alignment of the targeted sequence for CILP-M in human (SEQ ID NO: 21) with mouse (SEQ ID NO: 22), bovine (SEQ ID NO: 23), and rat (SEQ ID NO: 24). The target sequence is highlighted, and protease cleavage is marked with an arrow. B) Specificity of the CILP-M assay. Reactivity towards the standard peptide (SLNPDTGLWE) (SEQ ID NO: 1), truncated peptide (NPDTGLWE) (SEQ ID NO: 4), elongated peptide (WSLNPDTGLWE) (SEQ ID NO: 2) and non-sense standard peptide and coater (DSGPEYADVV) (SEQ ID NO: 16). Signals are shown as relative luminescence (RLU) per second, as a function of standard peptide.

FIG. 2: In vitro cleavage of human articular cartilage by enzymes. CILP-M levels were measured in human articular cartilage cleaved by MMP1, MMP2, MMP3, MMP8, MMP9, MMP10, MMP12, MMP13, ADAMTS4 and ADAMTS5 using the CILP-M competitive chemiluminescence immunoassay assay.

FIG. 3: Levels of CILP-M in the discovery and validation cohort and associated ROC curves. A) CILP-M levels in the discovery cohort including healthy donors (n=13), patients with rheumatoid arthritis (RA, n=18), patients with ankylosing spondylitis (AS, n=14) and patients with osteoarthritis (OA, n=8). B) ROC curve analysis evaluating the ability of CILP-M to discriminate between healthy controls and RA/OA/AS respectively, in the discovery cohort. C) CILP-M levels in the validation cohort including healthy donors (n=105), patients with RA (n=23) and patients with AS (n=89). D) ROC curve analysis evaluating the ability of CILP-M to discriminate between healthy controls and RA/AS respectively, in the validation cohort. Data was analysed using ANCOVA test (adjusting for age and gender in the validation cohort) and Area under the receiver operating characteristic curve (AUROC) analysis.

FIG. 4: Levels of CILP-M stratified into responders (n=26) and non-responders (n=9) to anti-TNF-α therapy. Response to anti-TNF-α treatment in patients with AS at baseline (week 0) and after 12 weeks. Data are shown as Tukey box plots and a logistic regression model was used to predict response to anti-TNF-α based on 50% reduction of BASDAI index after 3 months of treatment.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

All reagents used were high quality chemicals from Merck (Whitehouse Station, NJ, USA) and Sigma (St. Louis MO, USA) unless stated otherwise. All synthetic peptides used for antibody production and assay validation were purchased from Genscript (Piscataway, NJ, US) (Table 1).

TABLE 1
The sequences of the synthetic peptides used for
monoclonal antibody production, assay development and validation

| Peptide type | Sequence |
|---|---|
| Immunogenic peptide | SLNPDTGLWE-GGC-KLH* (SEQ ID NO: |
| | 17) |
| Selection peptide | SLNPDTGLWE (SEQ ID NO: 1) |
| Elongated selection peptide | WSLNPDTGLWE (SEQ ID NO: 2) |
| Truncated selection peptide | NPDTGLWE (SEQ ID NO: 4) |
| Non-sense coating and standard | DSGPEYADVV (SEQ ID NO: 16) |
| peptide | |

*Keyhole Limpet Hemocyanin

### Monoclonal Antibody Development, Production, and Characterization

The amino acid sequence 700'⬇SLNPDTGLWE'710 (SEQ ID NO: 1) was used for generation of monoclonal antibodies (mAbs). Immunization was initiated by subcutaneous injection of 200 μl emulsified antigen and 100 μg immunogenic peptide (SLNPDTGLWE-GGC-KLH) in 4- to 6-week-old Balb/C mice using Stimmune (Thermo Fisher). The immunizations were repeated every second week until stable serum antibody titer levels were reached. The mouse with the highest serum titer was selected for fusion and rested for a month. Subsequently, the mouse was boosted intravenously with 50 μg immunogenic peptide in 100 μl 0.9% NaCl solution 3 days before isolation of the spleen for cell fusion. To produce hybridoma cells, the mouse spleen cells were fused with SP2/0 myeloma cells as described by Gefter et al. (13). Subsequently, the clones were plated into 96-well microtiter plates for further growth, and the limiting dilution method was applied to promote monoclonal growth. An indirect ELISA performed on streptavidin-coated plates was used for the screening of supernatant reactivity. SLNPDTGLWE-K-Biotin (SEQ ID NO: 18) was used as screening peptide, while the standard peptide SLNPDTGLWE (SEQ ID NO: 1) was used to further test the specificity of the clones. Supernatant was collected from the hybridoma cells and purified using HiTrap affinity columns (GEHealthcare Life Science, Little Chalfront, Buckinghamshire, UK) according to manufacturer's instructions and antibody isotype was determined using Rapid ELISA Mouse monoclonal antibody Isotyping Kit (Invitrogen, Carlsbad, CA, USA) following the manufacturer's protocol.

Native reactivity was assessed using human serum, citrate plasma, heparin plasma, EDTA plasma and rat serum, purchased from a commercial supplier (Valley Biomedical, Winchester, VA). The mAb was selected to specifically recognize the standard peptide (SLNPDTGLWE (SEQ ID NO: 1)), and not an elongated or truncated sequence of one amino acid (WSLNPDTGLWE (SEQ ID NO: 2) and NPDTGLWE (SEQ ID NO: 14), respectively).

### CILP-M Assay Development

The development of the competitive chemiluminescence immunoassay (CLIA) included preliminary optimizing experiments where reagents, concentrations, incubation-time and -temperature were analyzed by several tests. The CILP-M competitive ELISA procedure was as follows: a 96-well streptavidin-coated white microplate (Greiner Bio-One, Kremsmünster, Austria) was coated with 2.5 ng/mL biotinylated synthetic peptide (SLNPDTGLWE-K-Biotin) (SEQ ID NO: 18) dissolved in assay buffer (10 mM phosphate buffered saline (PBS), 1% bovine serum albumin, 0.1% Tween-20, 0.36% Bronidox, 4 g/L NaCl, adjusted to pH 7.4 at 20 °C) and incubated for 30 min at 20 °C with constant shaking (300 rpm) in darkness. Next, 20 μL/well of standard peptide (100 ng/ML) and samples were added to the appropriate wells, followed by the addition of 100 μL/well of HRP-labelled antibody diluted in assay buffer to the concertation of 100 ng/ml and incubated for 1 hr at 20 °C with constant shaking (300 rpm) in darkness. After each incubation step, wells were washed five times with standard washing buffer (20 mM Tris, 50 mM NaCl, pH 7.2). The chemiluminescence substrate (Roche, BM Chemiluminescence ELISA substrate (POD), Basel, Switzerland) working solutions were mixed 15 min before use and 100 μL/well were added to plate and incubated for 3 min at 20 °C with constant shaking (300 rpm) in darkness. The relative light units were measured at all wavelengths within 5 min on a microplate luminometer reader (SpectraMax M5, Molecular Devices, CA, USA). A standard curve was plotted using a 4-parameter logistic curve fit Y=(A−D)/(1+(x/C)\hat{ }B)+D, where R>0.9. Data were analysed using the SoftMax Pro version 7.0.3 software

### Technical Evaluation

Two-fold dilutions of four human serum samples were used to assess linearity of the immunoassay. Linearity was calculated as a percentage of recovery of the undiluted sample. Antibody specificity was calculated as percentage of signal inhibition by two-fold diluted standard peptide (SLNPDTGLWE) (SEQ ID NO: 11), elongated peptide (WSLNPDTGLWE) (SEQ ID NO: 2), truncated peptide (LNPDTGLWE) (SEQ ID NO: 3), and non-sense peptide (DSGPEYADVV) (SEQ ID NO: 16). The intra- and inter-assay variation was determined by 10 independent runs of five quality controls and two kit controls run in double determinations. Accuracy of the assay was measured in healthy human serum samples spiked with standard peptide and a serum sample with a known high CILP-M concentration and calculated as the percentage recovery of the measured value and the expected concentration of the peptide or the serum sample with high CILP-M plus the concentration of the analyte in serum. Analytical interference was performed by adding a low/high content of haemoglobin (2.50/5 mg/mL), lipemia/lipids (1.50/5 mg/mL) and biotin (3/9 ng/ml) to a serum sample of known concentration. Recovery percentage was calculated with the normal serum sample as reference. The normal reference levels for haemoglobin, lipidaemia/lipids and biotin were 0-10 mg/dl (0-0.00161 mmol/L), <150 mg/dl (<1.6935 mmol/L) and 0.221-3.004 ng/ml, respectively. The interference was calculated as the percentage recovery of the analyte in non-spiked serum. The measurement range was defined as the range between lower limit of measurement range (LLMR) and the upper limit of measurement range (ULMR), which were determined from 10 independent runs with the standard peptide. Measurements below LLMR or above ULMR were assigned the value of LLMR/ULMR, respectively. IC₅₀ (half-maximal inhibition concentration) was determined from the standard curve. The analyte stability was examined through temperature tests and repeated freeze-thaw cycles of serum samples. The temperature tests included different time point and temperatures where CILP-M levels were measured in three human serum samples after 0, 2-, 4-, 24-, and 48-hours incubation at either 4 °C or 20 °C. The recovery was estimated with 0 hours sample as a reference. The effect of four repeated freeze/thaw cycles of three serum samples was assessed where freeze/thaw recovery was calculated with the zero cycle samples as a reference. Each sample were run in double determination.

### In Vitro Cleavage

Articular cartilage biopsies from OA patients who underwent knee replacement surgery were obtained from Gentofte Hospital Denmark. The collection and retrieval of the human cartilage complied with international ethical guidelines for handling human sample and patient information. All participants signed an informed consent, and the study was approved by the local ethical committee. A broad panel of enzymes, known to cleave human articular cartilage were tested (4). The enzymes used were MMP-1, MMP-2, MMP-3, MMP-8, MMP-9, MMP-10, MMP-12, MMP-13, ADAMTS4 and ADAMTS5. The cartilage cleavage was performed as follows; the pulverized cartilage sample (30 mg) was incubated with 1 μg of each enzyme with 250 μL digestion buffer as previously described (4). The digestion was carried out for 24 h in replicates. The reaction was stopped by adding 5 mM EDTA. Cleaved products were measured using the CILP-M ELISA.

### Biological Evaluation of the CILP-M ELISA

The biological utility of the CILP-M ELISA was evaluated in serum samples from a discovery and a validation cohort. The discovery cohort was acquired from the commercial vendor Proteogenex (Culver City, CA). The discovery cohort included a group of healthy donors (n=13), patients diagnosed with RA (n=18), patients diagnosed with AS (n=14), and patients diagnosed with OA (n=8). The validation cohort was collected at University of Alberta, Canada, and included serum samples from patients diagnosed with RA (n=23), patients diagnosed with AS (n=89), and results were compared with age-matched healthy donors from the commercial vendor BioIVT (Westerbury, NY, USA) (n=105). The Bath AS Disease Activity Index (BASDAI) and modified Stoke AS Spine Score (mSASSS) were monitored for each AS patient in the validation cohort. Samples from both cohorts were collected after informed consent and approval by the local Ethical Committee and in compliance with the Helsinki Declaration of 1975. Serum samples were obtained and stored at −80 °C until use.

### Ethical Statement

All animals were treated according to the guidelines for animal welfare. Monoclonal antibody production in mice was approved by the Danish National Authority (The Animal Experiments Inspectorate) under approval number 2013-15-2934-00956.

### Statistical Analysis

For all statistical analysis performed, a p-value below 0.05 was considered significant. Baseline characteristics are described as number (frequency) and percentage for categorical variables, and as mean (±SD) for continuous variables. Kruskal-Wallis rank test was used to examine baseline differences between groups of participants. One-way ANCOVA analyses were performed for both studies at baseline. Area under the receiver operating characteristic curve (AUROC) analysis was performed to investigate the discrimination accuracy of CILP-M between patients with AS or RA and healthy controls at baseline. The validation cohort analysis was adjusted for age and gender. A logistic regression model was used to predict response to anti-TNF-α treatment (Enbrel, Humira, Remicade or Simponi) in patients with AS based on 50% reduction of BASDAI index after 3 months of treatment in the validation study (patients were biologically naïve prior treatment).

Statistical analysis and graphs were performed using GraphPad Prism version 9 (GraphPad Software, Inc., La Jolla, CA) and R studio version 4.2.1 (R Foundation for Statistical Computing, Vienna, Austria. URL https://www.R-project.org).

### Results

### Technical Evaluation and Characterization of CILP-M

The monoclonal antibody clone NB326 #76 13B5-1C6-2C4 showed the best native reactivity, peptide affinity, and stability for the assay and was chosen for assay development. The isotype, sequence and CDRs of this monoclonal antibody were determined. The sequence of the chains are as follows (CDRs underlined and in bold; N-terminus signal peptide and C-terminus Constant region in italics):

### Heavy Chain Sequence (Mouse IgG1 Isotype)

(SEQ ID NO: 19)
MGWSRIFLFLLSIIAGVHQVQLQQSGPELVKPGASVRISCKASGYTFTNY
YIHWVKQRPGQGLEWIGWISPGIVDTEYNEKFKNKATLTADKSSRTAYMQ
LSSLTSEDSAVYFCARRSAGSYGDFDYWGQGTTLTVSSAKTTPPSVYPLA
PGSAAQTNSMVTLGCLVKGYFPEPVTVTWNSGSLSSGVHTFPAVLQSDLY
TLSSSVTVPSSTWPSETVTCNVAHPASSTKVDKKIVPRDCGCKPCICTVP
EVSSVFIFPPKPKDVLTITLTPKVTCVVVDISKDDPEVQFSWFVDDVEVH
TAQTQPREEQFNSTFRSVSELPIMHQDWLNGKEFKCRVNSAAFPAPIEKT
ISKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHNH
HTEKSLSHSPGK

### Light Chain Sequence (Mouse Kappa Isotype)

(SEQ ID NO: 20)
MESQTQVFVYMLLWLSGVDGDIVMTQSQKEMSTSVGDRVSVTCKASQNVG
TDVVWYQQRPGQSPKALMYSASYRYSGVPDRFTGSGSGTDFTLTISNVQS
EDLAEYFCQHYDNYPLTFGAGTKLELKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

A summary of the technical evaluation of the CILP-M assay can be found in Table 2. Briefly, the measurement range (LLOQ-ULOQ) was determined to 0.54-30.00 ng/ml. The inter- and intra assay variation was 10.3% and 7.2% respectively, and linearity was approved from undiluted to a 2-fold dilution in human serum. The stability of the analyte was acceptable both during prolonged storage of human serum samples at 4 °C and 20 °C (95% and 92%, respectively), and three freeze-thaw cycles (92-119%). Haemoglobin, lipemia and biotin did not interfere with measurements of CILP-M in human serum.

TABLE 2
Summary of technical parameters for CILP-M

| TECHNICAL VALIDATION | RESULTS | |
|---|---|---|
| IC₅₀ | 2.0 | ng/mL |
| Measurement range (LLOQ-ULOQ) | 0.54-30 | ng/mL |
| LLOB | 0.40 | ng/mL |
| Inter-assay variation | 10.3% | |
| Intra-assay variation | 7.2% | |
| Dilution of human serum | 1 + 0 or 1 + 1 | |
| Spiking recovery (serum in selection | 88-103% | |
| peptide) | | |
| Analyte recovery 48 h, 4 °C/20 °C | 95%/92% | |
| Analyte recovery, 3 freeze/thaw cycles | 92-119% | |
| Haemoglobin recovery (low/high) | 85%/75% | |
| Lipemia recovery (low/high) | 97%/97% | |
| Biotin interference | >100 | ng/mL |

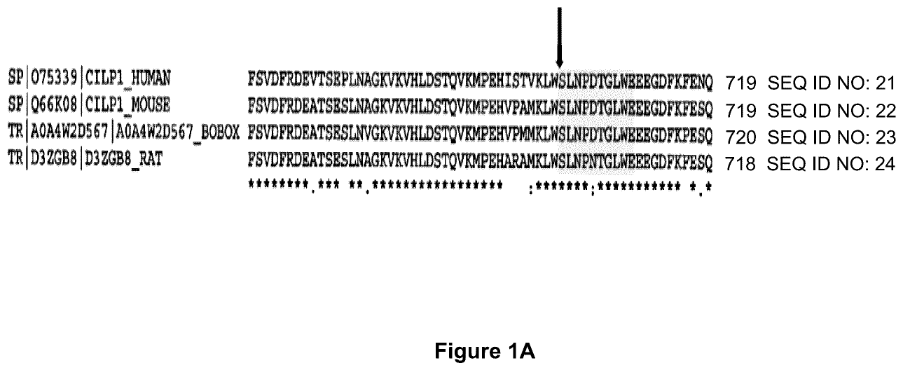

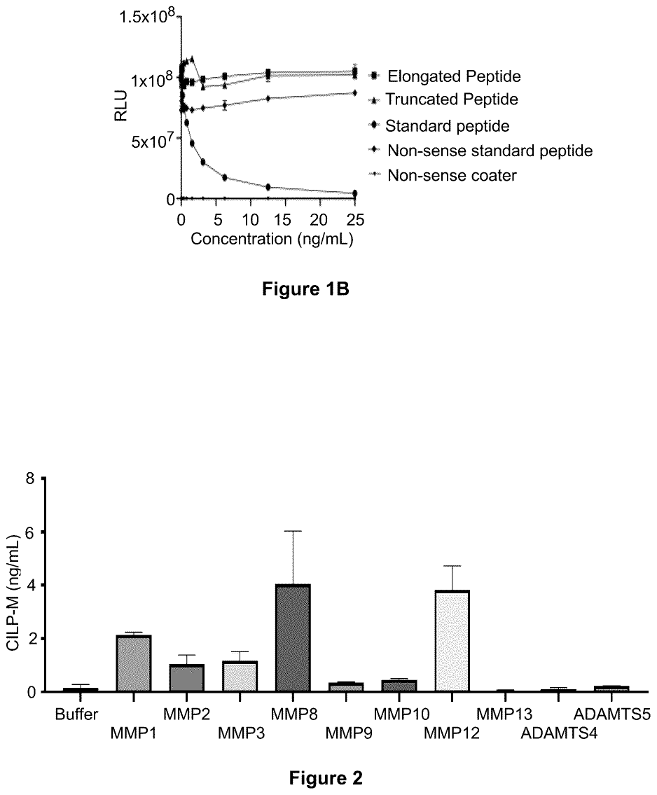

The human targeted sequence (SEQ ID NO: 21) for CILP-M was aligned with the corresponding mouse (SEQ ID NO: 22), bovine (SEQ ID NO: 23) and rat (SEQ ID NO: 24) sequences using UNIPROT, and the corresponding target sequence in mouse and bovine are 100% aligned with the human sequence, while the rat sequence has one mismatch in position 5 (FIG. 1A). To evaluate the specificity of the CILP-M assay, the mAb was tested towards the elongated peptide, truncated peptide, non-sense standard peptide and non-sense coater, and showed no reactivity towards those peptides (FIG. 1B).

To investigate the responsible enzyme for CILP-M generation, human articular cartilage was cleaved with a panel of enzymes. It was observed that CILP-M was primarily generated by MMP-1, MMP-8, and MMP-12 (FIG. 2).

### Baseline Demographic and Clinical Characteristics

Table 3 and 4 show the demographic characteristics of both cohorts. In the discovery cohort, a significant difference was found between the ages of the groups (p<0.0001). The significant difference is driven by patients with OA, which is on average 30 years older than healthy donors, RA and AS in this cohort. There was no difference in gender distribution between the groups. In the validation cohort, there were also a significant difference between the ages of the groups (p<0.0001), with RA patients being on average 15 years older than the healthy donors and AS patients. In addition, there were more men in the RA and AS groups compared to the healthy donors (p<0.0001).

TABLE 3
Patient Demographics for discovery cohort

| | | Rheumatoid | Ankylosing | Osteoar- | |
|---|---|---|---|---|---|
| | Healthy | arthritis | spondylitis | thritis | |
| | (N = 13) | (N = 18) | (N = 14) | (N = 8) | p-value |
| Age, | 45.08 | 35.72 | 35.71 | 69.88 | <0.001 |
| years | (8.97) | (3.30) | (3.10) | (3.31) | |
| Gender, | 6 | 9 | 8 | 4 | 0.952 |
| male | (46.2%) | (50.0%) | (57.1%) | (50.0%) | |
| BMI | — | 24.11 | 22.63 | 27.64 | 0.594 |
| | | (1.80) | (1.70) | (3.66) | |
| Ethnicity | | | | | |
| Unknown | 0 | 8 | 8 | 0 | |
| Caucasian | 12 | 10 | 6 | 8 | |
| | (92.3%) | (100.0%) | (100.0%) | (100.0%) | |
| Hispanic | 1 | 0 | 0 | 0 | |
| | (7.7%) | (0.0%) | (0.0%) | (0.0%) | |
| CILP-M, | 0.76 | 1.85 | 1.97 | 1.92 | <0.001 |
| ng/ml | (0.22) | (0.75) | (0.99) | (0.85) | |

Categorical variables are written as number (percentage), while continuous variables are mean (standard deviation). Kruskal-Wallis rank test was used to compare differences among the groups. Abbreviations: BMI, body mass index.

TABLE 4
Patient Demographics for validation cohort

| | | Rheumatoid | Ankylosing | |
|---|---|---|---|---|
| | Healthy | arthritis | spondylitis | |
| | (N = 105) | (N = 23) | (N = 89) | p-value |
| Age, years | 41.23 | 57.78 | 45.33 | <0.001 |
| | (12.69) | (10.94) | (12.62) | |
| Gender(male) | 53 | 18 | 68 | <0.001 |
| | (52.0%) | (78.3%) | (77.3%) | |
| Disease duration, | NA | 13.45 | 20.92 | |
| years | | (11.26) | (13.72) | |
| anti-TNF-α | | | | |
| Enbrel | NA | 14 | 20 | |
| | | (60.9%) | (22.5%) | |
| Humira | NA | NA | 11 | |
| | | | (12.4%) | |
| Remicade | NA | 9 | 29 | |
| | | (39.1%) | (32.6%) | |
| Simponi | NA | NA | 7 | |
| | | | (7.9%) | |
| No (NSAIDs) | NA | NA | 22 | |
| | | | (24.7%) | |
| Baseline CRP, mg/L | NA | 26.93 | 15.87 | |
| | | (38.86) | (21.26) | |
| Baseline ESR, | NA | 34.73 | 22.06 | |
| mm/hour | | (27.00) | (20.44) | |
| Baseline BASDAI | NA | NA | 5.64 | |
| | | | (2.36) | |
| Baseline mSASSS | NA | NA | 15.62 | |
| | | | (18.98) | |
| CILP-M, ng/mL | 1.06 | 2.78 | 2.67 | <0.001 |
| | (0.57) | (1.87) | (0.88) | |

Categorical variables are written as number (percentage), while continuous variables are mean (standard deviation). Kruskal-Wallis rank test was used to compare differences among the groups. Abbreviations: CRP, C-reactive protein; ESR, Erythrocyte sedimentation rate; BASDAI, Bath Ankylosing Spondylitis Disease Activity Index; mSASSS, modified Stoke Ankylosing Spondylitis Spinal Score

### CILP-M is Upregulated in Rheumatic Diseases in the Discovery and Validation Cohort

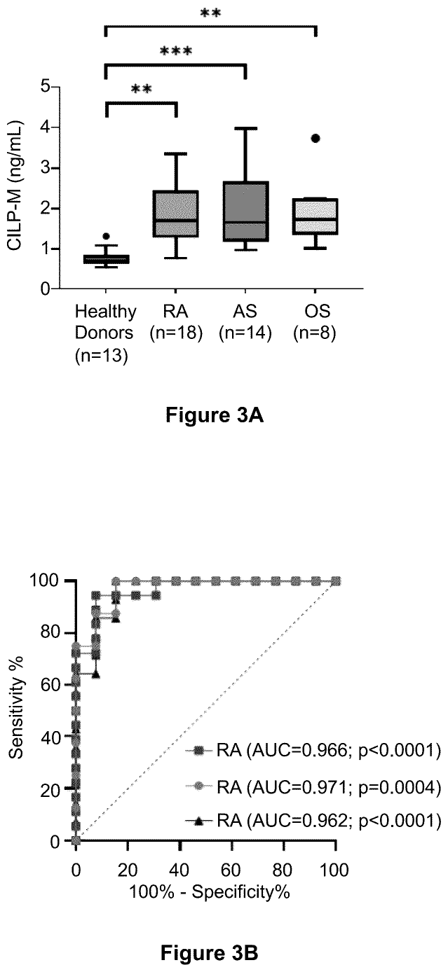

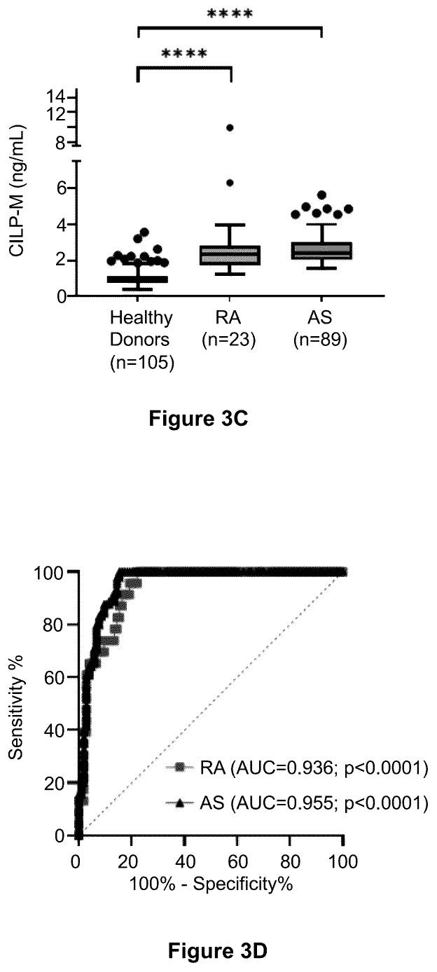

Levels of CILP-M were measured in two independent cohorts. The discovery cohort consisted of healthy donors, and patients diagnosed with RA, AS and OA. Here, patients with RA, AS and OA showed significantly higher levels of CILP-M compared to healthy donors (p=0.001 p=0.0007, and p=0.006, respectively, FIG. 3A). No difference was found between the patient groups. The diagnostic power (AUROC) of CILP-M for patients suffering from RA compared to healthy donors was 0.966 (95% CI=0.910-1.000, p<0.0001), for patients suffering from AS compared to healthy donors was 0.962 (95% CI=0.896-1.000, p<0.0001) and for patients suffering from OA compared to healthy donors was 0.971 (95% CI=0.911-1.000, p=0.0004), as shown in FIG. 3B.

The validation cohort included healthy donors, and patients diagnosed with either RA or AS. Here, patients with RA and AS confirmed higher levels of CILP-M in patients with rheumatic disease (both p<0.0001. FIG. 3C). The diagnostic power (AUROC) of CILP-M for patients suffering from RA compared to healthy donors was 0.936 (95% CI=0.894-0.978, p<0.0001), and for patients suffering from AS compared to healthy donors was 0.955 (95% CI=0.926-0.984, p<0.0001), as shown in FIG. 3D.

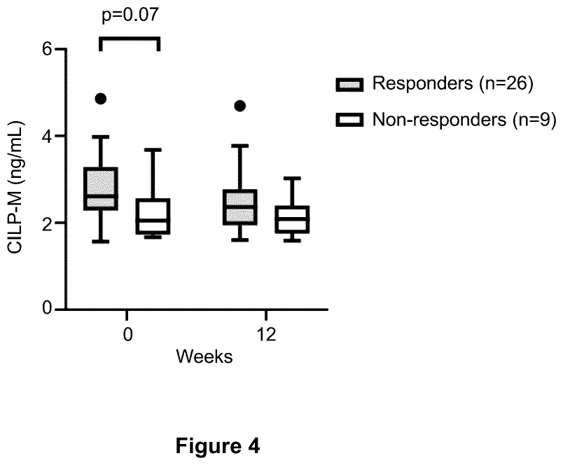

In the validation cohort, 67 patients with AS were treated with anti-TNF-α treatment and 35 patients out of the 67 had BASDAI measurements before and after treatment. To investigate response to treatment based on a 50% reduction of BASDAI, the 35 patients where BASDAI information was available had CILP-M levels measured. It was found that the likelihood of responding to anti-TNF-α treatment showed a tendency to be higher for a unit increase of CILP-M at baseline (OR=3.28, 95% CI=[0.85; 12.64] p-value=0.07, FIG. 4).

### Discussion

The applicant has developed and characterized a competitive ELISA for detection of CILP-M using a monoclonal antibody targeting the MMP-generated neo-epitope of CILP-1. The main findings from the Examples were: 1) A technically robust and specific assay towards the CILP-M neo-epitope, primarily generated by MMP1, MMP8 and MMP12, was developed; 2) CILP-M was measurable in human serum, and the levels of CILP-M were increased in patients with RA, AS and OA as shown in the discovery and validation cohort; and 3) CILP-M showed the ability to discern between responders and non-responded to anti-TNF-α treatment.

The CILP-M assay was characterised as a technically robust and accurate assay by showing acceptable dilution recovery, interference, and stability tests. The inter- and intra-variation was accepted with 10.3% and 7.2% respectively. The epitope CILP-M target was previously found by mass spectrometry of human articular cartilage (4), which was primarily generated by ADAMTS5. To confirm which protease generated the CILP-M cleavage site, a series of proteases were tested. In contrast with the previous finding, our in-vitro cleavages showed that CILP-M epitope was generated by MMP-1, MMP-8, and MMP-12. Destruction and fusion of articular cartilage is a key feature in rheumatic diseases (11-13). Serin proteases and MMP are highly expressed in the extracellular matrix when joint impairment is occurring and are responsible of cartilage degradation (13). ADAMTS4 and ADAMTS5 are considered the main aggrecan degrading enzymes in cartilage (3), whereas MMP can degrade all components of the ECM (14). MMP-1 and MMP-8 are two of the most classic collagenases that are most important for the degradation of collagen within cartilage (14). MMP-1 is produced primarily by synovial cells that edge the joints and has a predominant role in OA because it controls the process of collagen degradation (15). High levels of CILP-1 have been found in synovial tissue (10), which could be further cleaved by MMP-1. MMP-8 is expressed in sites of inflammation and has been associated with a wide range of inflammatory disorders (13, 16), whereas MMP-12 is secreted by inflammatory macrophages (17), and it has been found that increased MMP-12 expression in macrophages worsened the development of inflammatory arthritis in rabbits (18).

To the applicant's knowledge, this is the first study investigating the cleavage products of CILP-1 in rheumatic diseases and the first study detecting epitopes of CILP-1 in a blood-based sample. It was found that levels of CILP-M were increased in RA, AS and OA, compared to healthy donors. In previous studies, CILP-1 has been associated with cartilage degenerative diseases, especially OA (6,7). It has also been shown that anti-CILP antibodies are found in OA and RA patients, suggesting that the autoimmune response against CILP is related to the pathogenesis of OA and RA (19). To the applicant's knowledge, this was the first study investigating the role of CILP-1 in AS patients. The present findings are in line with previously studies that showed that patients with AS have a higher extracellular matrix remodelling in cartilage (20,21).

However, CILP-1 has also been related with pulmonary hypertension and cardiac fibrosis, suggesting is not only specific to articular cartilage (22,23).

As there is a need for better tools to monitor response to treatment in patients with AS, we also investigated if CILP-M was able to predict response to anti-TNF-α, and we observed that patients responding to anti-TNF-α treatment had higher levels of CILP-M prior to treatment.

In conclusion, a novel neo-epitope biomarker CILP-M, measuring a MMP-1-, MMP-8- and MMP-12-mediated fragment of CILP-1 was developed and validated for use in human serum samples. CILP-M was upregulated in patients with RA and AS in two independent cohorts. Based on these results, CILP-M is useful to assess cartilage remodelling in joint degenerative diseases.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Sophia Fox A J, Bedi A, Rodeo S A. The Basic Science of Articular Cartilage: Structure, Composition, and Function. Sports Health [Internet] SAGE Publications; 2009 [cited 2022 Mar. 9]; 1:461. Available from:/pmc/articles/PMC3445147/
2. Lorenzo P, Bayliss M T, Heinegård D. A Novel Cartilage Protein (CILP) Present in the Mid-zone of Human Articular Cartilage Increases with Age*. 1998 [cited 2022 Jul. 19]; Available from: http://www.jbc.org
3. Goldring M B, Marcu K B. Cartilage homeostasis in health and rheumatic diseases. Arthritis Res Ther [Internet] BioMed Central; 2009 [cited 2022 Jul. 19]; 11:224. Available from:/pmc/articles/PMC2714092/
4. Zhen E Y, Brittain I J, Laska D A, Mitchell P G, Sumer E U, Karsdal M A, et al. Characterization of metalloprotease cleavage products of human articular cartilage. Arthritis Rheum [Internet] Arthritis Rheum; 2008 [cited 2022 Jul. 19]; 58:2420-31. Available from: https://pubmed.ncbi.nlm.nih.gov/18668564/
5. Lorenzo P, Bayliss M T, Heinegård D. Altered patterns and synthesis of extracellular matrix macromolecules in early osteoarthritis. Matrix Biol 2004; 23:381-91.
6. Valdes A M, Hart D J, Jones K A, Surdulescu G, Swarbrick P, Doyle D V., et al. Association study of candidate genes for the prevalence and progression of knee osteoarthritis. Arthritis Rheum [Internet] John Wiley & Sons, Ltd; 2004 [cited 2022 Jul. 19]; 50:2497-507. Available from: https://onlinelibrary.wiley.com/doi/full/10.1002/art.20443
7. Valdes A M, Van Oene M, Hart D J, Surdulescu G L, Loughlin J, Doherty M, et al. Reproducible genetic associations between candidate genes and clinical knee osteoarthritis in men and women. Arthritis Rheum [Internet] John Wiley & Sons, Ltd; 2006 [cited 2022 Jul. 19]; 54:533-9. Available from: https://onlinelibrary.wiley.com/doi/full/10.1002/art.21621
8. Seki S, Kawaguchi Y, Chiba K, Mikami Y, Kizawa H, Oya T, et al. A functional SNP in CILP, encoding cartilage intermediate layer protein, is associated with susceptibility to lumbar disc disease. Nat Genet [Internet] Nature Publishing Group; 2005 [cited 2022 Jul. 19]; 37:607-12. Available from: https://u-toyama. elsevierpure. com/en/publications/a-functional-snp-in-cilp-encoding-cartilage-intermediate-layer-pr
9. Tsuruha J I, Masuko-Hongo K, Kato T, Sakata M, Nakamura H, Nishioka K, et al. Implication of Cartilage Intermediate Layer Protein in Cartilage Destruction in Subsets of Patients With Osteoarthritis and Rheumatoid Arthritis. ARTHRITIS Rheum 2001; 44:838-45.
10. Kerna I, Kisand K, Tamm A, Tamm A. Cartilage intermediate layer protein is produced in synovial membrane of osteoarthritic joint and upregulated in osteoarthritis associated fibrosis. Bone Abstr [Internet] BioScientifica; 2013 [cited 2022 Jul. 19]; 1. Available from: http://www.bone-abstracts.org/ba/0001/ba0001pp15
11. Park D R, Kim J, Kim G M, Lee H, Kim M, Hwang D, et al. Osteoclast-associated receptor blockade prevents articular cartilage destruction via chondrocyte apoptosis regulation. Nat Commun 2020 111 [Internet] Nature Publishing Group; 2020 [cited 2022 Jul. 19]; 11:1-11. Available from: https://www.nature.com/articles/s41467-020-18208-y
12. Bleil J, Sieper J, Maier R, Schlichting U, Hempfing A, Syrbe U, et al. Cartilage in facet joints of patients with ankylosing spondylitis (AS) shows signs of cartilage degeneration rather than chondrocyte hypertrophy: Implications for joint remodeling in AS. Arthritis Res Ther [Internet] BioMed Central Ltd.; 2015 [cited 2021 May 31]; 17. Available from:/pmc/articles/PMC4506408/
13. Rannou F, François M, Corvol M-T, Berenbaum F. Cartilage breakdown in rheumatoid arthritis. 2005 [cited 2022 Jul. 19]; Available from: http://france.elsevier.com/direct/BONSOI/
14. Mehana E-SE, Khafaga A F, El-Blehi S S. The role of matrix metalloproteinases in osteoarthritis pathogenesis: An updated review. 2019;
15. Kuzmiński A, Przybyszewski M, Graczyk M, Bartuzi Z. The role of extracellular matrix metalloproteinases and their inhibitors in allergic diseases. Postep Dermatologii i Alergol 2012; 29:384-9.
16. Van Lint P, Libert C. Matrix metalloproteinase-8: Cleavage can be decisive. Cytokine Growth Factor Rev 2006; 17:217-23.
17. Chen Y E. MMP-12, An Old Enzyme Plays a New Role in the Pathogenesis of Rheumatoid Arthritis? Am J Pathol [Internet] American Society for Investigative Pathology; 2004 [cited 2022 Aug. 3]; 165:1069. Available from:/pmc/articles/PMC1618647/
18. Wang X, Liang J, Koike T, Sun H, Ichikawa T, Kitajima S, et al. Overexpression of Human Matrix Metalloproteinase-12 Enhances the Development of Inflammatory Arthritis in Transgenic Rabbits. Am J Pathol [Internet] American Society for Investigative Pathology; 2004 [cited 2022 Aug. 3]; 165:1375. Available from:/pmc/articles/PMC1618618/
19. Tsuruha J I, Masuko-Hongo K, Kato T, Sakata M, Nakamura H, Nishioka K. Implication of cartilage intermediate layer protein in cartilage destruction in subsets of patients with osteoarthritis and rheumatoid arthritis. Arthritis Rheum 2001; 44:838-45.
20. Hušáková M, Bay-Jensen A C, Forejtová Š, Zegzulková K, Tomčík M, Gregová M, et al. Metabolites of type I, II, III, and IV collagen may serve as markers of disease activity in axial spondyloarthritis. Sci Rep 2019; 9:1-10.
21. Maksymowych W P. Biomarkers for diagnosis of axial spondyloarthritis, disease activity, prognosis, and prediction of response to therapy. Frontiers in Immunology Frontiers Media S.A.; 2019.
22. Keranov S, Dörr O, Jafari L, Troidl C, Liebetrau C, Kriechbaum S, et al. CILP1 as a biomarker for right ventricular maladaptation in pulmonary hypertension. [cited 2022 Jul. 29]; Available from: https://doi.org/10.1183/13993003.01192-2019
23. Zhang C-L, Zhao Q, Liang H, Qiao X, Wang J-Y, Wu D, et al. Cartilage intermediate layer protein-1 alleviates pressure overload-induced cardiac fibrosis via interfering TGF-31 signaling. 2018 [cited 2022 Jul. 12]; Available from: https://doi.org/10.1016/j.yjmcc.2018.02.006

1. A monoclonal antibody that specifically binds to the N-terminus amino acid sequence SLNPDTGLWE (SEQ ID NO: 1).

2. The monoclonal antibody of claim 1, wherein the monoclonal antibody does not specifically bind to a peptide having the N-terminus amino acid sequence WSLNPDTGLWE (SEQ ID NO: 2).

3. The monoclonal antibody of claim 1, wherein the monoclonal antibody does not specifically bind to a peptide having the N-terminus amino acid sequence LNPDTGLWE (SEQ ID NO: 3).

4. The monoclonal antibody of claim 1, wherein the monoclonal antibody does not specifically bind to a peptide having the N-terminus amino acid sequence NPDTGLWE (SEQ ID NO: 4).

5. The monoclonal antibody claim 1, wherein the monoclonal antibody is raised against a synthetic peptide having the N-terminus amino acid sequence SLNPDTGLWE (SEQ ID NO: 1).

6. A method of immunoassay comprising:
i) contacting a patient sample with a monoclonal antibody that specifically binds to the N-terminus amino acid sequence SLNPDTGLWE (SEQ ID NO: 1);
ii) detecting and determining the amount of binding between said monoclonal antibody and peptides in the sample.

7. The method of claim 6, wherein the method is a method for detecting and/or monitoring a disease in a patient and/or assessing the likelihood of the patient responding to a treatment with a drug to treat a disease, the method further comprising;
iii) correlating said amount of binding with values associated with normal healthy subjects and/or values associated with known disease severity and/or values obtained from said patient at a previous time point and/or with a predetermined cut-off value.

8. The method of claim 7, wherein the disease is a joint disease.

9. The method of claim 8, wherein the joint disease is rheumatoid arthritis, ankylosing spondylitis or osteoarthritis.

10. The method of claim 7, wherein the treatment with a drug to treat a disease is an anti-TNF-α treatment.

11. The method of claim 6, wherein the monoclonal antibody does not specifically bind to a peptide having the N-terminus amino acid sequence WSLNPDTGLWE (SEQ ID NO: 2).

12. The method of claim 6, wherein the monoclonal antibody does not specifically bind to a peptide having the N-terminus amino acid sequence LNPDTGLWE (SEQ ID NO: 3).

13. The method of claim 6, wherein the monoclonal antibody does not specifically bind to a peptide having the N-terminus amino acid sequence NPDTGLWE (SEQ ID NO: 4).

14. The method of claim 6, wherein the monoclonal antibody is raised against a synthetic peptide having the N-terminus amino acid sequence SLNPDTGLWE (SEQ ID NO: 1).

15. The method of claim 6, wherein the patient sample is selected from blood, serum or plasma.

16. The method of claim 6, wherein the immunoassay is a competition assay or a sandwich assay.

17. The method of claim 6, wherein the immunoassay is a radio-immunoassay or an enzyme-linked immunosorbent assay.

18. An immunoassay kit comprising a monoclonal antibody that specifically binds to the N-terminus amino acid sequence SLNPDTGLWE (SEQ ID NO: 1), and at least one of:
a streptavidin coated well plate;
a biotinylated peptide SLNPDTGLWE-L-Biotin (SEQ ID NO: 15), wherein L is an optional linker;
a secondary antibody for use in a sandwich immunoassay;
a calibrator protein comprising the N-terminus amino acid sequence SLNPDTGLWE (SEQ ID NO: 1);
an antibody biotinylation kit;
an antibody HRP labelling kit;
an antibody radiolabelling kit; or
an assay visualisation kit.

19. The immunoassay kit of claim 18, wherein the monoclonal antibody does not specifically bind to a peptide having the N-terminus amino acid sequence WSLNPDTGLWE (SEQ ID NO: 2).

20. The immunoassay kit of claim 18, wherein the monoclonal antibody does not specifically bind to a peptide having the N-terminus amino acid sequence LNPDTGLWE (SEQ ID NO: 3).

21. The immunoassay kit of claim 18, wherein the monoclonal antibody does not specifically bind to a peptide having the N-terminus amino acid sequence NPDTGLWE (SEQ ID NO: 4).

22. The immunoassay kit of claim 18, wherein the monoclonal antibody is raised against a synthetic peptide having the N-terminus amino acid sequence SLNPDTGLWE (SEQ ID NO: 1).


SEQUENCE LISTING (24 sequences)

SEQ ID NO: 1 - protein, 10 aa, synthetic construct
SLNPDTGLWE

SEQ ID NO: 2 - protein, 11 aa, synthetic construct
WSLNPDTGLWE

SEQ ID NO: 3 - protein, 9 aa, synthetic construct
LNPDTGLWE

SEQ ID NO: 4 - protein, 8 aa, synthetic construct
NPDTGLWE

SEQ ID NO: 5 - protein, 11 aa, synthetic construct
KASQNVGTDVV

SEQ ID NO: 6 - protein, 7 aa, synthetic construct
SASYRYS

SEQ ID NO: 7 - protein, 9 aa, synthetic construct
QHYDNYPLT

SEQ ID NO: 8 - protein, 5 aa, synthetic construct
NYYIH

SEQ ID NO: 9 - protein, 17 aa, synthetic construct
WISPGIVDTEYNEKFKN

SEQ ID NO: 10 - protein, 11 aa, synthetic construct
RSAGSYGDFDY

SEQ ID NO: 11 - protein, 74 aa, synthetic construct
KASQNVGTDVVWYQQRPGQSPKALMYSASYRYSGVPDRFTGSGSGTDFTLTISNVQSEDLAEYFCQHYDNYPLT

SEQ ID NO: 12 - protein, 79 aa, synthetic construct
NYYIHWVKQRPGQGLEWIGWISPGIVDTEYNEKFKNKATLTADKSSRTAYMQLSSLTSEDSAVYFCARRSAGSYGDFDY

SEQ ID NO: 13 - protein, 107 aa, synthetic construct
DIVMTQSQKFMSTSVGDRVSVTCKASQNVGTDVVWYQQRPGQSPKALMYSASYRYSGVPDRFTGSGSGTDFTLTISNVQSEDLAEYFCQHYDNYPLTFGAGTKLELK

SEQ ID NO: 14 - protein, 120 aa, synthetic construct
QVQLQQSGPELVKPGASVRISCKASGYTFTNYYIHWVKQRPGQGLEWIGWISPGIVDTEYNEKFKNKATLTADKSSRTAYMQLSSLTSEDSAVYFCARRSAGSYGDFDYWGQGTTLTVSS

SEQ ID NO: 15 - protein, 10 aa, synthetic construct
  MOD_RES 10: note = biotinylated with optional linker
SLNPDTGLWE

SEQ ID NO: 16 - protein, 10 aa, synthetic construct
DSGPEYADVV

SEQ ID NO: 17 - protein, 13 aa, synthetic construct
  MOD_RES 13: note = linked to Keyhole Limpet Hemocyanin
SLNPDTGLWEGGC

SEQ ID NO: 18 - protein, 11 aa, synthetic construct
  MOD_RES 11: note = biotinylated
SLNPDTGLWEK

SEQ ID NO: 19 - protein, 463 aa, synthetic construct
MGWSRIFLFLLSIIAGVHCQVQLQQSGPELVKPGASVRISCKASGYTFTNYYIHWVKQRPGQGLEWIGWISPGIVDTEYNEKFKNKATLTADKSSRTAYMQLSSLTSEDSAVYFCARRSAGSYGDFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 234 aa, synthetic construct
MESQTQVFVYMLLWLSGVDGDIVMTQSQKFMSTSVGDRVSVTCKASQNVGTDVVWYQQRPGQSPKALMYSASYRYSGVPDRFTGSGSGTDFTLTISNVQSEDLAEYFCQHYDNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 60 aa, Homo sapiens
FSVDFRDEVTSEPLNAGKVKVHLDSTQVKMPEHISTVKLWSLNPDTGLWEEEGDFKFENQ

SEQ ID NO: 22 - protein, 60 aa, Mus musculus
FSVDFRDEATSESLNAGKVKVHLDSTQVKMPEHVPAMKLWSLNPDTGLWEEEGDFKFESQ

SEQ ID NO: 23 - protein, 60 aa, Bos taurus
FSVDFRDEATSESLNVGKVKVHLDSTQVKMPEHVPMMKLWSLNPDTGLWEEEGDFKPESQ

SEQ ID NO: 24 - protein, 60 aa, Rattus rattus
FSVDFRDEATSESLNAGKVKVHLDSTQVKMPEHARAMKLWSLNPNTGLWEEEGDFKFESQ